(19) United States
(12) Patent Application Publication
(10) Pub. No.: US 2025/0135176 A1
(43) Pub. Date: May 1, 2025

(54) QUICK CONNECT, RAPID FLUID TRANSFER CONNECTION

(71) Applicant: Terumo BCT, Inc., Lakewood, CO (US)

(72) Inventors: James LADTKOW, Broomfield, CO (US); Kestas PARAKININKAS, Englewood, CO (US)

(21) Appl. No.: 18/383,674

(22) Filed: Oct. 25, 2023

(51) Int. Cl. A61M 39/10; A61M 39/00; A61M 39/24; F16K 15/04

(52) U.S. Cl. CPC A61M 39/1011; A61M 39/24; F16K 15/044; A61M 2039/0027; A61M 2039/1027; A61M 2039/242; A61M 2039/248; A61M 2205/583

(57) ABSTRACT

A quick connect, rapid fluid transfer connection includes a first connector having a first end and an opposing second end and a second connector having a first end and an opposing second end, where the second end of the second connector is configured to receive at least a portion of the second end of the first connector. At least one of the first and second connectors includes a check valve. For example, the first connector may include a cavity and a ball movable therewithin. The cavity having a first portion with a first average diameter and a second portion with a second average diameter. The ball having a third average diameter that is smaller than the first average diameter and greater than the second average diameter.

### GOVERNMENT FUNDING

This invention was made with government support under contract number W81XWH-15-9-00001 awarded by the Medical Technology Enterprise Consortium (MTEC). The government may have certain rights in this invention.

### FIELD

The present disclosure relates to a quick connect, rapid fluid transfer connection, for example, for use during the reconstitution of a blood component, where the connection includes a first connector and a second connector, at least one of the first and second connectors having a check valve.

### BACKGROUND

This section provides background information related to the present disclosure which is not necessarily prior art.

Connectors or fittings are used to fluidly join different bags or container, for example, to effectuate reconstitution of freeze-dried materials, such as dried plasma. Reconstitution of freeze-dried materials often includes connecting a first bag or container and a second bag or container and moving a liquid material from the first bag or container to the second bag or container holding the freeze-dried material. Once the fluid transfer is complete it is important to disconnect and/or stop flow between the first bag or container and the second bag or container to prevent backflow into the second bag or container. In the instance of the reconstitution of dried plasma, backflow not only causes loss of product but may create a state where therapy may not be safely rendered, for example, because the concentration of the reconstituted plasma becomes unknown, thereby altering intended dosages. Accordingly, it would be desirable to develop systems and methods for using the same that prevent or eliminates the risks of backflow when connecting different bags or containers and moving materials from a first bag or container to a second bag or container.

### SUMMARY

This section provides a general summary of the disclosure and is not a comprehensive disclosure of its full scope or all of its features.

The present disclosure provides a first example quick connect, rapid fluid transfer connection.

In at least one example embodiment, the quick connect, rapid fluid transfer connection includes a first connector fluidly associated with a first bag and a second connector fluidly associated with a second bag. One of the first and second connectors includes a check valve.

In at least one example embodiment, the first connector further includes a cavity having a first portion with a first average diameter and a second portion with a second average diameter, and the check valve includes a ball movable within the cavity. The ball may have a third average diameter that is smaller than the first average diameter and greater than the second average diameter.

In at least one example embodiment, the first connector further includes a retention component disposed within and extending along at least a portion of the cavity.

In at least one example embodiment, the first connector includes a first plunger assembly including a first plunger and a first spring that are movable between a first position and a second position.

In at least one example embodiment, the first plunger is disposed upstream of the check valve.

In at least one example embodiment, the second connector includes a second plunger assembly including a second plunger and a second spring that are movable between a first position and a second position. The second plunger may be configured to engage with the first plunger and to move the first plunger from its first position to its second position when joined to the first connector to form the connection.

In at least one example embodiment, the first connector includes a first end and an opposing second end. The first end of the first connector may be fluidly associated with the first bag. The second connector may include a first end and an opposing second end. The first end of the second connector may be fluidly associated with the second bag. The second end of the second connector may be configured to receive at least a portion of the second end of the first connector.

In at least one example embodiment, the first connector includes a first hub and a second hub. The first and second hubs may include one or more guide components to assist in and ensure proper alignment of the first and second hubs when joined to define the first connector.

In at least one example embodiment, the second connector includes a first hub and a second hub. The first and second hubs may include one or more guide components to assist in and ensure proper alignment of the first and second hubs when joined to define the first connector.

In at least one example embodiment, the first bag includes a blood component, and the second bag includes an aqueous solution.

The present disclosure provides a second example quick connect, rapid fluid transfer connection.

In at least one example embodiment, the quick connect, rapid fluid transfer connection includes a first connector having a first end and an opposing second end and a second connector having a first end and an opposing second end, where the second end of the second connector is configured to receive at least a portion of the second end of the first connector. The first connector may include a cavity and a ball movable therewithin. The cavity may have a first portion with a first average diameter and a second portion with a second average diameter. The ball may have a third average diameter that is smaller than the first average diameter and greater than the second average diameter.

In at least one example embodiment, the first connector includes a first plunger assembly including a first plunger and a first spring that are movable between a first position and a second position. The second connector may include a second plunger assembly including a second plunger and a second spring that are movable between a first position and a second position. The second plunger may be configured to engage with the first plunger and to move the first plunger from its first position to its second position when joined to the first connector to form the connection.

In at least one example embodiment, the first connector includes a first hub and a second hub. The first and second hubs may include one or more first guide components to assist in and ensure proper alignment of the first and second hubs when joined to define the first connector. The second connector may include a third hub and a fourth hub. The third and fourth hubs may include one or more second guide components to assist in and ensure proper alignment of the third and fourth hubs when joined to define the first connector.

The present disclosure provides a method for reconstituting a blood component.

In at least one example embodiment, the method includes joining a first connector and a second connector, where the first connector is fluidly associated with a first bag, and the second connector fluidly is fluidly associated with a second bag. At least one of the first and second connectors includes a check valve.

In at least one example embodiment, the first connector further includes a cavity having a first portion with a first average diameter and a second portion with a second average diameter, the check valve includes a ball movable within the cavity. The ball may have a third average diameter that is smaller than the first average diameter and greater than the second average diameter.

In at least one example embodiment, the first connector further includes a retention component disposed within and extending along at least a portion of the cavity.

In at least one example embodiment, the first connector includes a first plunger assembly including a first plunger and a first spring that are movable between a first position and a second position. The second connector may include a second plunger assembly including a second plunger and a second spring that are movable between a first position and a second position. The second plunger may be configured to engage with the first plunger and to move the first plunger from its first position to its second position when joined to the first connector to form the connection.

In at least one example embodiment, the first connector includes a first end and an opposing second end. The first end of the first connector may be fluidly associated with the first bag. The second connector may include a first end and an opposing second end. The first end of the second connector may be fluidly associated with the second bag. The second end of the second connector may be configured to receive at least a portion of the second end of the first connector.

In at least one example embodiment, the first connector may include a first hub and a second hub. The first and second hubs may include one or more guide components to assist in and ensure proper alignment of the first and second hubs when joined to define the first connector.

In at least one example embodiment, the second connector may include a first hub and a second hub. The first and second hubs may include one or more guide components to assist in and ensure proper alignment of the first and second hubs when joined to define the first connector.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1 is a perspective view of an example connection, for example, for use during the reconstitution of a blood component, where the connection includes a first connector joined to a second connector, the first connector having a check valve in accordance with at least one example embodiment of the present disclosure;

FIG. 2 is a cross-sectional view of the connection illustrated in FIG. 1 where the check valve is positioned to allow movement through the connection in accordance with at least one example embodiment of the present disclosure;

FIG. 3 is a cross-sectional view of the connection illustrated in FIG. 1 where the check valve is positioned to prevent movement through the connection in accordance with at least one example embodiment of the present disclosure;

FIG. 4 is a cross-sectional, perspective view of the connection illustrated in FIG. 1 where the check valve is positioned to allow movement through the connection in accordance with at least one example embodiment of the present disclosure;

FIG. 5 is a cross-sectional, perspective view of the connection illustrated in FIG. 1 where the check valve is positioned to prevent movement through the connection in accordance with at least one example embodiment of the present disclosure

FIG. 6 is a perspective view of the connection illustrated in FIG. 1 where the first and second connectors are aligned but disconnected;

FIG. 7 is a perspective view of the first connector, where a first end of the first connector includes a cap in accordance with at least one example embodiment of the present disclosure;

FIG. 8 is a cross-sectional view of the first connector illustrated in FIG. 7;

FIG. 9 is a cross-sectional, perspective view of the first connector illustrated in FIG. 7;

FIG. 10 is another perspective view of the first connector, where the first end of the first connector includes the cap, and the second end of the first connector is joined to a bag or container in accordance with at least one example embodiment of the present disclosure;

FIG. 11 is a perspective view of the second connector, where a first end of the second connector includes a cap in accordance with at least one example embodiment of the present disclosure;

FIG. 12 is a cross-sectional view of the second connector illustrated in FIG. 11;

FIG. 13 is a cross-sectional, perspective view of the second connector illustrated in FIG. 11;

FIG. 14 is another perspective view of the second connector, where the first end of the second connector includes the cap, and the second end of the second connector is joined to a bag or container in accordance with at least one example embodiment of the present disclosure; and

FIG. 15 is another perspective view of the connection illustrated in FIG. 1 where a first end of the connection is joined to a first bag or container and a second end of the connection is joined to a second bag or container in accordance with at least one example embodiment of the present disclosure.

Corresponding reference numerals indicate corresponding parts throughout the several views of the drawings.

### DETAILED DESCRIPTION

Example embodiments will now be described more fully with reference to the accompanying drawings.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected, or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers, and/or sections, these elements, components, regions, layers, and/or sections should not be limited by these terms. These terms may be only used to distinguish one element, component, region, layer or section from another region, layer, or section. Terms such as “first,” “second,” and other numerical terms when used herein do not imply a sequence or order unless clearly indicated by the context. Thus, a first element, component, region, layer, or section discussed below could be termed a second element, component, region, layer, or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

Various components are referred to herein as “operably associated.” As used herein, “operably associated” refers to components that are linked together in operable fashion and encompasses embodiments in which components are linked directly, as well as embodiments in which additional components are placed between the linked components. “Operably associated” components can be “fluidly associated.” “Fluidly associated” refers to components that are linked together such that fluid can be transported between them. “Fluidly associated” encompasses embodiments in which additional components are disposed between the two fluidly associated components, as well as components that are directly connected. Fluidly associated components can include components that do not contact fluid but contact other components to manipulate the system (e.g., a peristaltic pump that pumps fluids through flexible tubing by compressing the exterior of the tube).

Example embodiments will now be described more fully with reference to the accompanying drawings.

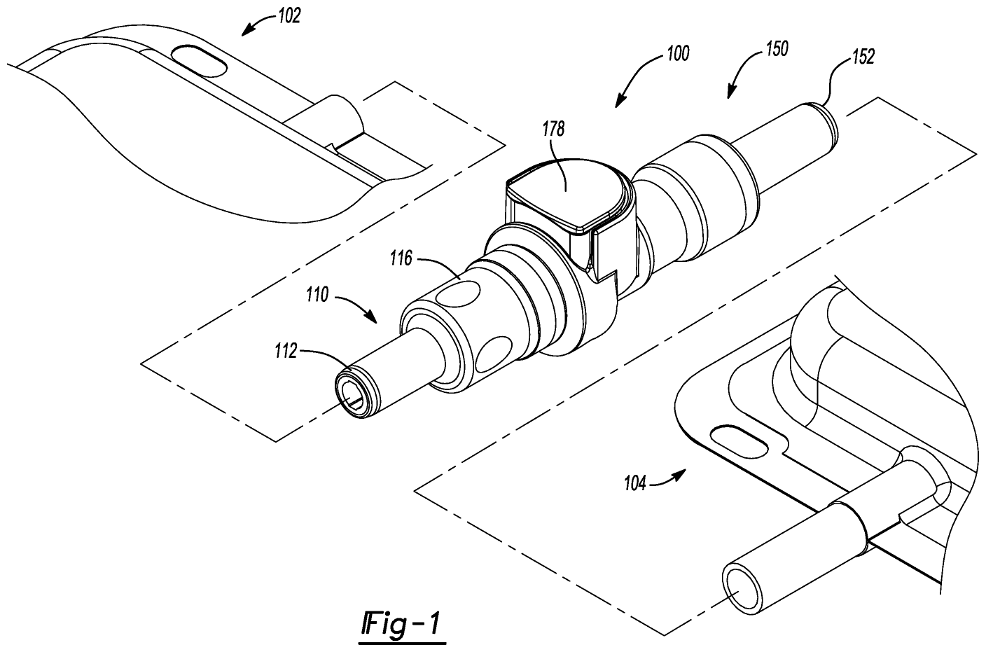

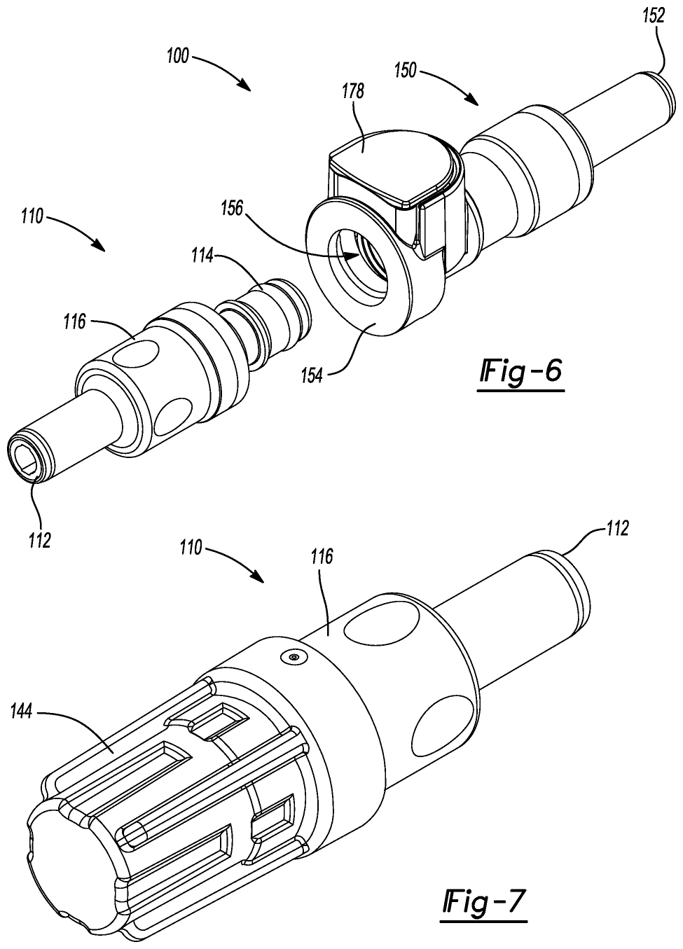

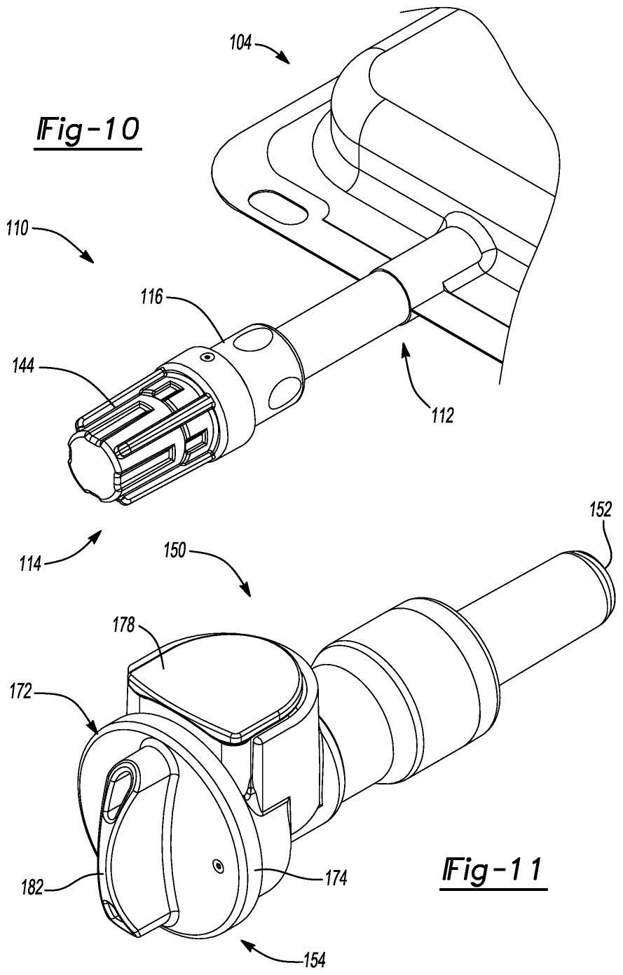

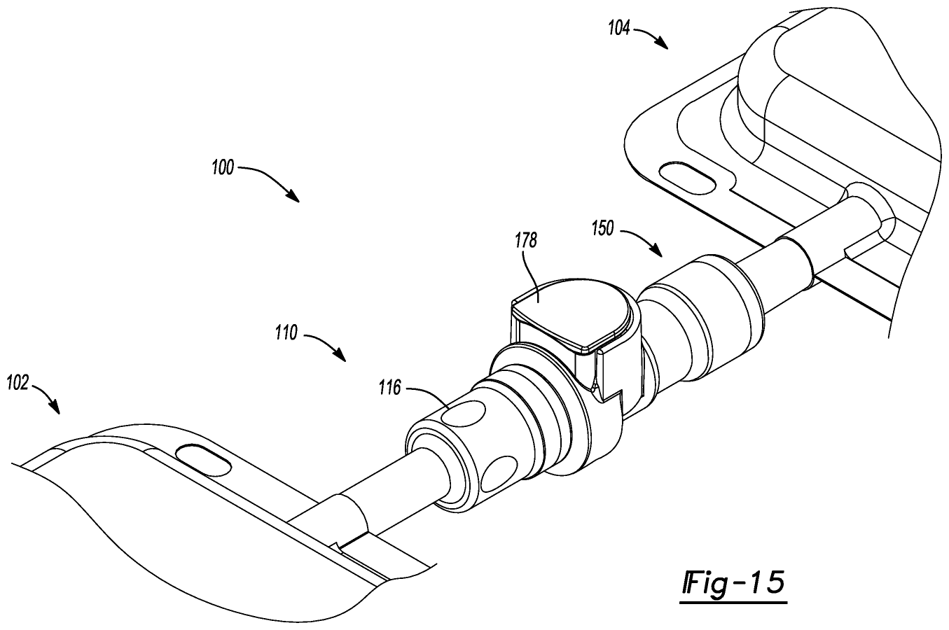

FIGS. 1-15 provide different views of an example connection (for example, a quick connect, rapid fluid transfer connection) 100 that may be used, for example, during the reconstitution of a blood component. As illustrated, the connection 100 includes a first connector (which may also be referred to as a first half and/or a first portion and/or a first component and/or male connector) 110 and a second connector (which may also be referred to as a second half and/or a second portion and/or a second component and/or female connector) 150.

The first connector 110 has a first end 112 configured to be fluidly associated with a first bag (which may also be referred to as a first container) 102 and a second end 114 that opposes the first end 112 and that is configured to be joined to the second connector 112. In at least one example embodiment, a first tube length may fluidly associate (for example, join together) the first connector 110 and the first bag 102. For example, the first end 112 of the first connector 110 may be configured to be received by an end of the first tube length. In at least one example embodiment, the first tube length may be integrally formed with the first bag 102.

The second connector 150 has a first end 152 configured to be fluidly associated with a second bag (which may also be referred to as a second container) 104 and a second end 154 that opposes the first end 152 and that is configured to be joined to the first connector 112. For example, as illustrated, for example, in FIGS. 1-6, at least a portion of the first connector 110 may be received within a first cavity (which may also be referred to as a first channel) 156 of the second connector 150. In at least one example embodiment a second tube length may fluidly associate (for example, join together) the second connector 150 and the second bag 104. For example, the first end 152 of the second connector 150 may be configured to be received by an end of the second tube length. In at least one example embodiment, the second tube length may be integrally formed with the second bag 104.

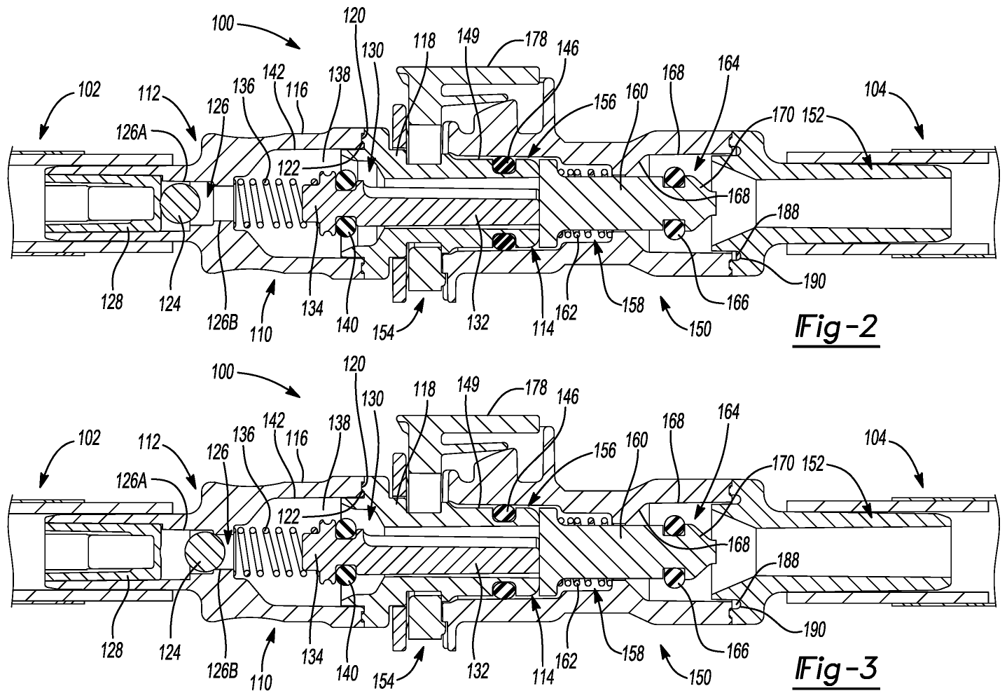

The first connector 110 includes a first hub (which may also be referred to as an outer component and/or an outer hub) 116 that defines the first end 112 of the first connector 110 and a second hub (which may also be referred to as an inner component and/or an inner hub) 118 that defines the second end 114 of the first connector 110. The first and second hubs 116, 118 join together to define the first connector 110. For example, in at least one example embodiment, as illustrated, for example, in FIGS. 2-5 and 8-9, the first hub 116 may be configured to receive at least a portion of the second hub 118. The first and second hubs 116, 118 may have corresponding guide components to assist in and ensure proper alignment of the first and second hubs 116, 118 when joined to define the first connector 110. For example, in at least one example embodiment, the second hub 118 may include one or more projections 120 and the first hub 116 may include one or more corresponding cavities 122 for receiving the projections 120.

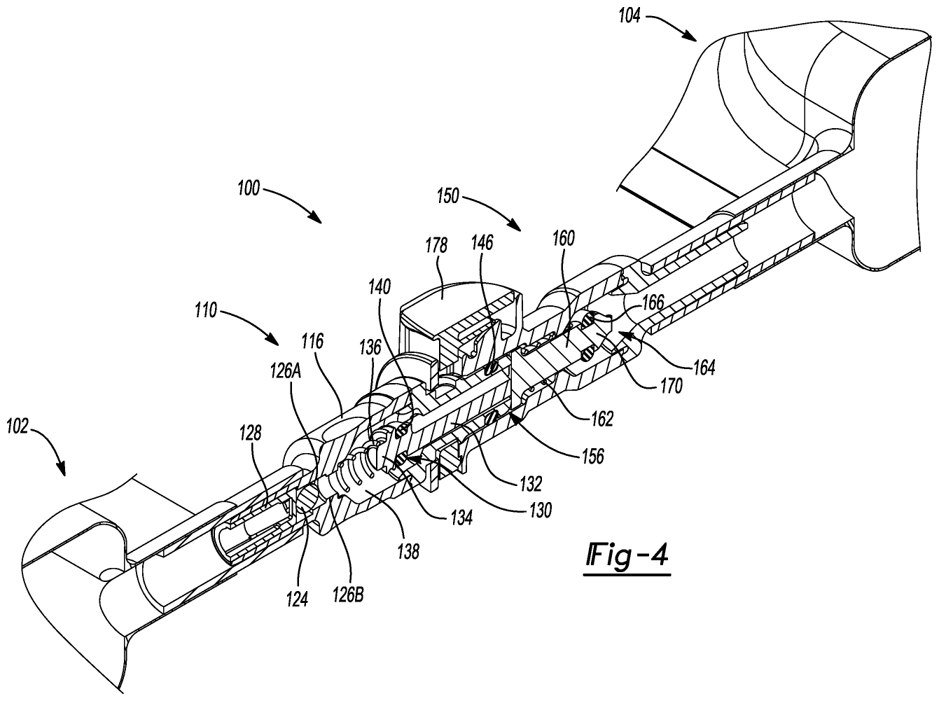

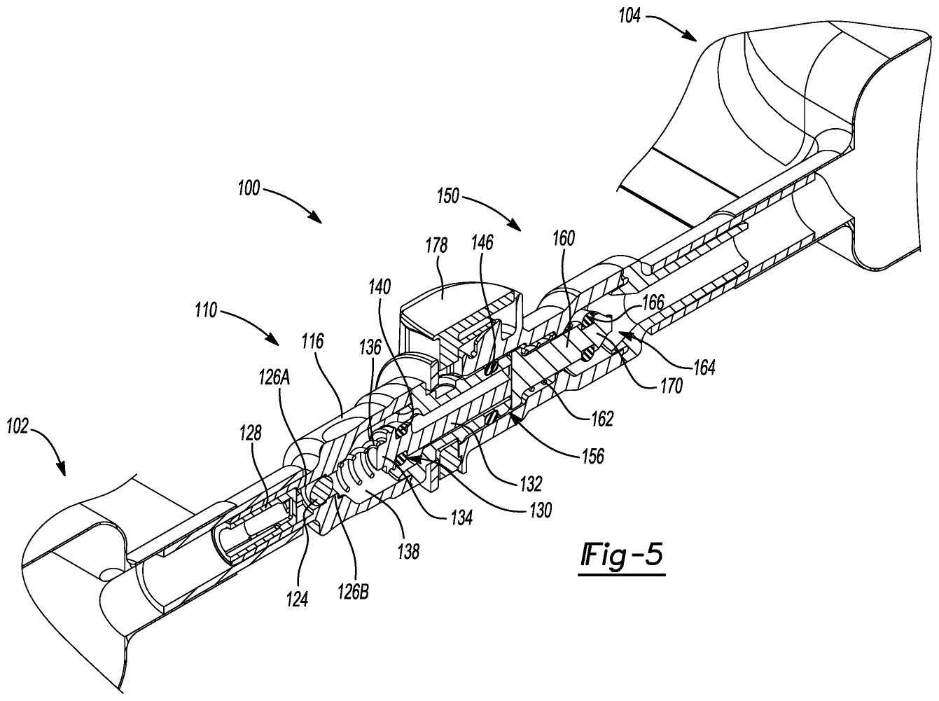

The first connector 110 includes a check valve configured to prevent backflow from the second bag 104 as connected to the second connector 112 through the connection 100 and into the first bag 102. In at least one example embodiment, the check valve may be a ball-check valve, where movement of a ball 124 within a second cavity (which may also be referred to as a second channel) 126 having two or more regions or portions 126A, 126B having different average diameters allows movement in a first direction while preventing movement in a second direction. For example, the second region or portion 126B of the second cavity 126 has a smaller average diameter than the ball 124, such that when the ball 124 is positioned near the second region 126B (for example, by fluid movement from the first end 112 in the direction of the second end 114) the ball 124 blocks the second cavity 126 and flow therethrough, as illustrated, for example, in FIGS. 3 and 5. In contrast, when a pressure is applied on the ball 124 from the second end 114 (for example, by fluid movement from the second end 114 in the direction of the first end 112), the ball 124 is caused to be positioned near the first region 126A which has a larger diameter than the ball 124 allowing for fluid to move therearound, as illustrated, for example, in FIGS. 2 and 4. In at least one example embodiment, a retention component 128 may be disposed in the second cavity 126. The retention component 128 may be disposed to prevent movement of the ball 124 along a remaining length of the second cavity 126.

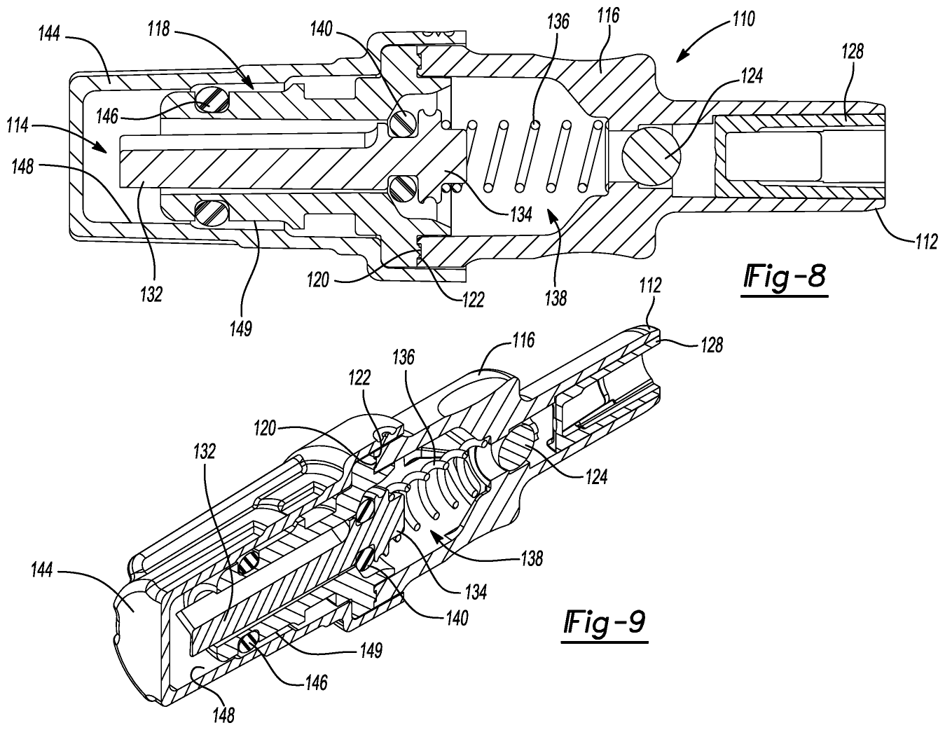

The first connector 110 includes a first spring loaded plunger 130 disposed, for example, upstream of the check valve (i.e., nearer to the second end 114 of the first connector 110 than the check valve). In at least one example embodiment, the first spring loaded plunger 130 includes a first plunger 132 having a first end 134 that is in communication with a first spring 136. The first plunger 132 and first spring 136 are movable between a first position (which may also be referred to as a starting position and/or an extended position) (see, e.g., FIGS. 8 and 9) and a second position (which may also be referred to as a stopping position and/or a retracted position) (see, e.g., FIGS. 2-5) within a third cavity 138. The third cavity 138 extends, for example, between, and is defined by, the first and second hubs 116, 118. In at least one example embodiment, the first connector 110 may include one or more first sealing members 140 configured to seal against an interior-facing wall or surface 142 defining at least a portion of the third cavity 138 to close the cavity 138 when the first plunger 132 is in the first position. For example, in at least one example embodiment, the one or more first sealing members 140 may be disposed on or near the first end 134 of the first plunger 132. In at least one example embodiment, the one or more first sealing members 140 may include one or more o-rings.

In at least one example embodiment, the first connector 110 includes a cap 144 that is configured to at least partially cover the second end 114 of the first connector 110, for example, to maintain a sterile state of the second end 114 prior to use, such as during storage and/or transport. In at least one example embodiment, as illustrated, for example, in FIGS. 8 and 9, the cap 144 may be configured to cover an entirety of the second hub 118 and at least part of the first hub 116. In at least one example embodiment, the cap 144 may be joined to the first connector 110 using one or more one-time use features. For example, the cap 144 may be joined to the first connector 110 using one or more ultrasonic stakes or welds. The one-time use features may prevent the cap 144 from being re-installed on the first connector 110 as a tamper evident feature to prevent contamination. In at least one example embodiment, the cap 114 may be removed from the first connector 110 by applying a twisting force.

In at least one example embodiment, the first connector 110 may include one or more second sealing members 146 configured to seal against an interior-facing wall or surface 148 of the cap 144 to help maintain the sterility of the second end 114 of the first connector 110. For example, in at least one example embodiment, the one or more second sealing members 146 may be disposed on or in an outer-facing wall or surface 149 of the second hub 118. In at least one example embodiment, the one or more second sealing members 146 may include one or more o-rings.

Like the first connector 110, in at least one example embodiment, the second connector 150 may include a first hub 184 that defines at least a portion the first end 152 of the second connector 150 and a second hub 186 that defines at least a portion of the second end 154 of the second connector 150. The first and second hubs 184, 186 join together to define the second connector 150. In at least one example embodiments, as illustrated, for example, in FIGS. 2-5 and 12-13, the first and second hubs 184, 186 may have corresponding guide components to assist in and ensure proper alignment of the first and second hubs 184, 186 when joined to define the second connector 150. For example, in at least one example embodiment, the second hub 186 may include one or more projections 188 and the first hub 184 may include one or more corresponding cavities 190 for receiving the projections 188.

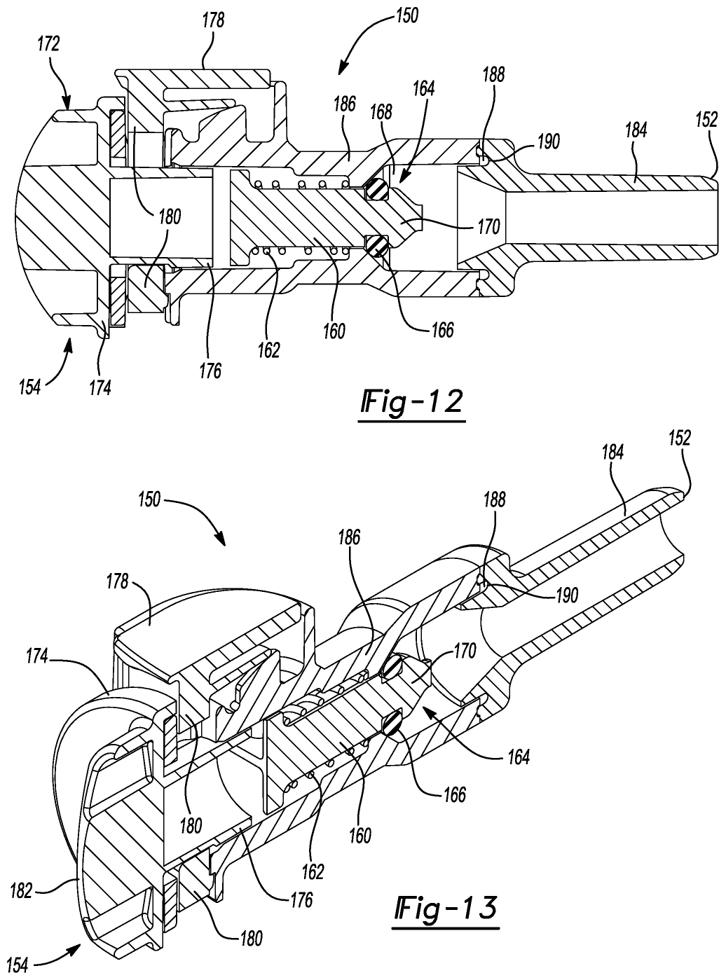

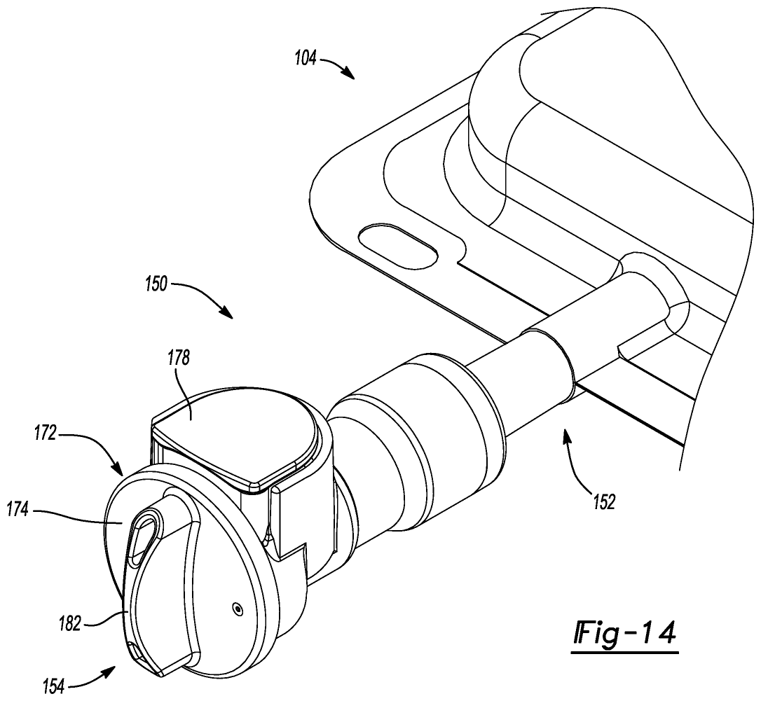

Also, like the first connector 110, in at least one example embodiment, the second connector 150 may include a (second) spring loaded plunger 158. The second spring loaded plunger 158 may include a second plunger 160 and a second spring 162 that surrounds at least a portion of the second plunger 160. The second plunger 160 and the second spring 162 are movable between a first position (which may also be referred to as a starting position and/or an extended position) (see, e.g., FIGS. 12 and 13) and a second position (which may also be referred to as a stopping position and/or a retracted position) (see, e.g., FIGS. 2-5) within a fourth cavity 164. In at least one example embodiment, the second connector 150 may include one or more second sealing members 164 configured to seal against an interior-facing wall or surface 168 defining at least a portion of the fourth cavity 164 to close the cavity 164 when the second plunger 160 is in the first position. For example, in at least one example embodiment, the one or more second sealing members 164 may be disposed on or near the first end 170 of the second plunger 160. In at least one example embodiment, the one or more second sealing members 164 may include one or more o-rings.

In at least one example embodiment, for example, during storage and/or transport, the second connector 150 includes a plug 172 that is configured to be received by the first cavity 156 and also to cover at least a portion of the second end 154 of the second connector 150. The plug 172 may be configured to help maintain a sterile state of the second end 154. In at least one example embodiment, as illustrated, for example, in FIGS. 12 and 13, the plug 172 may include a cap portion 174 and a projection 176 extending therefrom, where the cap portion 174 covers the at least a portion of the second end 154 of the second container 150, and the projection 176 is received by the first cavity 156. In at least one example embodiment, the second end 154 of the second connector 150 may include a button 178 that is configured to hold together the first and second connectors 110, 150. The button 178 may be a spring-loaded locking mechanism. For example, as illustrated, the button 178 may be in communication with one or more extensions 180 that extend towards the cavity 156.

The plug 172 may be removed from the second connector 150 by pulling the plug 172 away from the second connector 150 while also applying a pressure to the button 178. In at least one example embodiment, the plug 172 may include a handle 182 allowing the plug 172 to be pulled away from the second connector 150. As in the instance of the first connector 110 and the cap 144, in at least one example embodiment, the plug 172 may be joined to the second connector 150 via one or more one-time use features. For example, the plug 172 may be joined to the second connector 150 using one or more ultrasonic stakes or welds. The one-time use features may prevent the plug 172 from being re-installed on the second connector 150 as a tamper evident feature to prevent contamination.

When joined to define the connection 100, the first plunger 132 of the first connector 110 is configured to engage with the second plunger 160 of the second connector 150, as illustrated, for example, in FIGS. 2-5. For example, when the first and second connectors 110, 150 are joined, the second plunger 160 of the second connector 150 depresses first plunger 132 of the first connector 110 allowing for fluid movement between therebetween, and more particularly, because of the check valve, from the second connector 150 (and the second bag 104 attached thereto) to the first connector 110 (and the first bag 102 attached thereto). In the instances of reconstitution of a blood component, the first bag 102 may be configured to hold or carry the blood component, and the second bag 108 may be configured to hold or carry a reconstituting liquid, like an aqueous solution. In at least one example embodiment, the blood component may be freeze-dried blood component, such as dried plasma.

In various aspects, the present disclosure provides methods for reconstituting blood components, including, for example, freeze-dried plasma, using, for example, the connection 100 illustrated in FIGS. 1-15. Generally, the methods include joining together a first connector (like the first connector 110) and a second connector (like the second connector 150).

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A quick connect, rapid fluid transfer connection comprising:
a first connector fluidly associated with a first bag and including a check valve; and
a second connector fluidly associated with a second bag.

2. The connection of claim 1, wherein the first connector further includes a cavity having a first portion with a first average diameter and a second portion with a second average diameter, and the check valve includes a ball movable within the cavity, the ball having a third average diameter that is smaller than the first average diameter and greater than the second average diameter.

3. The connection of claim 2, wherein the first connector further includes a retention component disposed within and extending along at least a portion of the cavity.

4. The connection of claim 1, wherein the first connector includes a first plunger assembly including a first plunger and a first spring that are movable between a first position and a second position.

5. The connection of claim 4, wherein the first plunger is disposed upstream of the check valve.

6. The connection of claim 4, wherein the second connector includes a second plunger assembly including a second plunger and a second spring that are movable between a first position and a second position, the second plunger configured to engage with the first plunger and to move the first plunger from its first position to its second position when joined to the first connector to form the connection.

7. The connection of claim 1, wherein the first connector includes a first end and an opposing second end, the first end of the first connector being fluidly associated with the first bag, the second connector including a first end and an opposing second end, the first end of the second connector being fluidly associated with the second bag, and the second end of the second connector configured to receive at least a portion of the second end of the first connector.

8. The connection of claim 1, wherein the first connector includes a first hub and a second hub, the first and second hubs including one or more guide components to assist in and ensure proper alignment of the first and second hubs when joined to define the first connector.

9. The connection of claim 1, wherein the second connector includes a first hub and a second hub, the first and second hubs including one or more guide components to assist in and ensure proper alignment of the first and second hubs when joined to define the first connector.

10. The connection of claim 1, wherein the first bag includes a blood component, and the second bag includes an aqueous solution.

11. A quick connect, rapid fluid transfer connection comprising:
a first connector having a first end and an opposing second end, the first connector including a cavity and a ball movable therewithin, the cavity having a first portion with a first average diameter and a second portion with a second average diameter, and the ball having a third average diameter that is smaller than the first average diameter and greater than the second average diameter; and
a second connector having a first end and an opposing second end, the second end of the second connector configured to receive at least a portion of the second end of the first connector.

12. The connection of claim 11, wherein the first connector includes a first plunger assembly including a first plunger and a first spring that are movable between a first position and a second position, the second connector includes a second plunger assembly including a second plunger and a second spring that are movable between a first position and a second position, and the second plunger configured to engage with the first plunger and to move the first plunger from its first position to its second position when joined to the first connector to form the connection.

13. The connection of claim 11, wherein the first connector includes a first hub and a second hub, the first and second hubs including one or more first guide components to assist in and ensure proper alignment of the first and second hubs when joined to define the first connector; and
the second connector includes a third hub and a fourth hub, the third and fourth hubs including one or more second guide components to assist in and ensure proper alignment of the third and fourth hubs when joined to define the first connector.

14. A method for reconstituting a blood component, the method comprising:
joining a first connector and a second connector, the first connector fluidly associated with a first bag and including a check valve, and the second connector fluidly associated with a second bag.

15. The method of claim 14, wherein the first connector further includes a cavity having a first portion with a first average diameter and a second portion with a second average diameter, and the check valve includes a ball movable within the cavity, the ball having a third average diameter that is smaller than the first average diameter and greater than the second average diameter.

16. The method of claim 15, wherein the first connector further includes a retention component disposed within and extending along at least a portion of the cavity.

17. The method of claim 14, wherein the first connector includes a first plunger assembly including a first plunger and a first spring that are movable between a first position and a second position, the second connector includes a second plunger assembly including a second plunger and a second spring that are movable between a first position and a second position, and the second plunger configured to engage with the first plunger and to move the first plunger from its first position to its second position when joined to the first connector to form the connection.

18. The method of claim 14, wherein the first connector includes a first end and an opposing second end, the first end of the first connector being fluidly associated with the first bag, the second connector including a first end and an opposing second end, the first end of the second connector being fluidly associated with the second bag, and the second end of the second connector configured to receive at least a portion of the second end of the first connector.

19. The method of claim 14, wherein the first connector includes a first hub and a second hub, the first and second hubs including one or more guide components to assist in and ensure proper alignment of the first and second hubs when joined to define the first connector.

20. The method of claim 14, wherein the second connector includes a first hub and a second hub, the first and second hubs including one or more guide components to assist in and ensure proper alignment of the first and second hubs when joined to define the first connector.
